(19) United States
(12) Patent Application Publication
(10) Pub. No.: US 2017/0252306 A1
(43) Pub. Date: Sep. 7, 2017

(54) Pharmaceutical Compositions for Preventing and Treating Th1 or Th2 mediated Immune Disease Comprising 4H3MC(4-Hydroxy-3-methoxycinnamaldehyde) as an Active Ingredient

(71) Applicant: GWANGJU INSTITUTE OF SCIENCE AND TECHNOLOGY, Gwangju (KR)

(72) Inventors: Chang-Duk JUN, Gwangju (KR); Uroos Akber, Gwangju (KR); Hyun Su LEE, Gwangju (KR)

(21) Appl. No.: 15/397,877

(22) Filed: Jan. 4, 2017

(30) Foreign Application Priority Data
Jan. 4, 2016 (KR) 10-2016-0000518

(51) Int. Cl. A61K 31/11; A23L 33/10; A61K 9/00

(52) U.S. Cl. CPC A61K 31/11; A61K 9/0056; A23L 33/10; A23V 2002/00

(57) ABSTRACT

The present invention relates to a pharmaceutical composition for preventing or treating Th1- or Th2-mediated disease, which comprises 4H3MC (4-hydroxy-3-methoxycinnamaldehyde) as an active ingredient. 4H3MC (4-hydroxy-3-methoxycinnamaldehyde) according to the present invention shows the effect of inhibiting T-cell activity without inducing T-cell death. Thus, 4H3MC (4-hydroxy-3-methoxycinnamaldehyde) according to the present invention can be developed into an effective therapeutic agent against Th1- or Th2-mediated disease, etc.

### BACKGROUND OF THE INVENTION

Field of the Invention

The present invention relates to a pharmaceutical composition for preventing or treating Th1- or Th2-mediated immune disease, which comprises 4H3MC (4-hydroxy-3-methoxycinnamaldehyde) as an active ingredient.

### DESCRIPTION OF THE RELATED ART

T cell-mediated immune responses known as adaptive immunity develop antigen-specific T cells for eradiation of pathogens and malignant cells. In addition, T cell-mediated immune responses can also include atypical recognition of autoantigens, leading to autoimmune diseases [1]. These autoreactive lead to target organ and tissue damages, which are exaggerated by elevated T cell cytokines even after antigen clearance [2]. Thus, modulation of T cell responses is a central approach to develop autoimmune disease therapeutic agents.

When T cells are stimulated by antigen-presenting cells, the T cells are activated and a signal is transmitted into the cells. In this process, protein kinase C (PKC) plays an important role. Among PKC isotypes, particularly PKCα and PKCθ play functionally important roles. It was reported that PKCθ is very important in the production of IL-2 required for T cell proliferation and that PKCθ-deficient mice are defective in NF-κB activation. Furthermore, T cells require PKCα for IFN-γ production and cell proliferation [4-7]. Thus, PKC isotypes in T cells are regarded as an important target in the development of autoimmune disease therapeutic agents. However, a therapeutic agent against T-cell-mediated disease, which targets PKC, has not yet been developed.

Meanwhile, lignin is known to play an important role in the establishment of plant's resistance to biological and non-biological stresses. Cinnamic acid is believed to be a very important precursor for the biosynthesis of lignin, including 4H3MC (4-hydroxy-3-methoxycinnamaldehyde) as an intermediate derivative [8-9]. 4H3MC was reported to have antimicrobial activity and to show cytotoxicity against various cancer cells. Furthermore, 4H3MC was reported to more effectively inhibit melanin production in mouse melanoma cells and human melanocytes compared to other derivatives [10-12]. Meanwhile, the present inventors recently have found that 4-hydroxycinnamic acid (HCA), a structural analogue of cinnamic acid, inhibits T cell functions by blocking PKC and MAP kinase pathways, suggesting that other derivatives of cinnamic acid have potency to inhibit T cell activity [13]. However, the effect of 4H3MC, a cinnamic acid derivative, on the activation of T cells, has not yet been found.

Throughout the specification, a number of publications and patent documents are referred to and cited. The disclosure of the cited publications and patent documents is incorporated herein by reference in its entirety to more clearly describe the state of the related art and the present disclosure.

### DETAILED DESCRIPTION

### Technical Problem

The present inventors have studied and made efforts to develop a substance capable of preventing or treating Th1- or Th2-mediated immune disease by inhibiting T cell activity. As a result, the present inventors have found that 4H3MC (4-hydroxy-3-methoxycinnamaldehyde) can inhibit T cell activity by inhibiting the protein kinase C (PKC) activity of T cells and can be used to treat Th1 or Th2-mediated immune disease, thereby completing the present invention.

It is an object of the present invention to provide a pharmaceutical composition for preventing or treating Th1- or Th2-mediated immune disease, which comprises 4H3MC (4-hydroxy-3-methoxycinnamaldehyde) as an active ingredient.

Another object of the present invention is to provide a functional food composition or cosmetic composition for preventing or alleviating Th1- or Th2-mediated immune disease, which comprises, as an active ingredient, 4H3MC (4-hydroxy-3-methoxycinnamaldehyde) contained in a Curcuma longa extract.

Other objects and advantages of the present invention will be more apparent from the following description of the invention, the appended claims and the accompanying drawings.

### Technical Solution

In accordance to one aspect of the present invention, there is provided a pharmaceutical composition for preventing or treating Th1- or Th2-mediated immune disease, which comprises 4H3MC (4-hydroxy-3-methoxycinnamaldehyde) as an active ingredient.

The present inventors have made efforts to develop a substance capable of preventing or treating Th1- or Th2-mediated immune disease by inhibiting T-cell activity without inducing T-cell death. As a result, the present inventors have found that 4H3MC (4-hydroxy-3-methoxycinnamaldehyde) can inhibit T cell activity by inhibiting the protein kinase C (PKC) activity of T cells and also exhibits excellent therapeutic effects, particularly in animal models having atopic skin disease.

The composition of the present invention comprises 4H3MC (4-hydroxy-3-methoxycinnamaldehyde) as an active ingredient. 4H3MC (4-hydroxy-3-methoxycinnamaldehyde), a cinnamic acid derivative, is known to have antimicrobial activity and to show cytotoxicity against various cancer cells. However, the effects of 4H3MC on the inhibition of T-cell activity and the treatment of immune diseases have not yet been reported.

As used herein, the term “Th1-mediated immune disease” refers to a disease in which a cytokine (for example, IL-1β, IL-2, IL-12, IL-17, IFN-γ or TNF-α) which is produced by the production and/or activation of Th1 cells is involved.

As used herein, the term “Th1 cells” refers to a subset of helper T lymphocytes which are specified in terms of gene expression, protein secretion and functional activity. For example, Th1 cells show expression patterns of cytokines, including IL-2 and IFN-γ, but do not express cytokines, including IL-4, IL-5, IL-10 and IL-13. Th1 cells are involved in cell-mediated immune responses to various intracellular pathogens, organ-specific autoimmune diseases, and delayed-type hypersensitivities.

Th1-mediated immune disease to which the composition of the present invention is applied is not particularly limited. Specifically, the composition of the present invention is applied to transplant rejection, autoimmune disease, or inflammatory diseases. More specifically, Th1-mediated immune disease to which the composition of the present invention is applied is autoimmune disease or inflammatory disease.

Examples of Th1-mediated immune disease to which the composition of the present invention is applied include, but are not limited to, colitis, inflammatory bowel disease, type 1 diabetes, type 2 diabetes, rheumatoid arthritis, reactive arthritis, osteoarthritis, psoriasis, scleroderma, osteoporosis, atherosclerosis, myocarditis, endocarditis, pericarditis, cystic fibrosis, Hashimoto thyroiditis, Graves' disease, Hansen's disease, syphilis, Lyme disease, borreliosis, neuroborreliosis, tuberculosis, sarcoidosis, lupus, discoid lupus, chilblain lupus, lupus nephritis, systemic Lupus erythematous, asthma, macular degeneration, uveitis, irritable bowel syndrome, Crohn's disease, Sjögren's syndrome, fibromyalgia, chronic fatigue syndrome, chronic fatigue/immune dysfunction syndrome, myalgic encephalomyelitis, amyotrophic lateral sclerosis, Parkinson's disease, multiple sclerosis, autism spectrum disorder, attention deficit disorder, and attention-deficit hyperactivity disorder.

As used herein, the term “Th2-mediated immune disease” refers to a disease in which the IgE and mast cells caused by the production and activation of allergen-specific Th2 cells are involved.

As used herein, the term “Th2 cells” refers to a subset of helper T lymphocytes which are specified in terms of gene expression, protein secretion and functional activity. For example, Th2 cells shows expression patterns of cytokines, including IL-4, IL-5, IL-10 and IL-13. Th2 cells are involved in humoral immune responses.

Th2-mediated immune disease to which the composition of the present invention is applied is not particularly limited. Specifically, the composition of the present invention is applied to allergic disease.

Examples of Th2-mediated immune disease to which the composition of the present invention is applied include, but are not limited to, atopic dermatitis, other dermatological diseases associated with atopy, allergic rhinitis (acute or chronic), hay fever, and allergic bronchial asthma.

According to one embodiment of the present invention, the pharmaceutical composition of the present invention, which comprises 4H3MC as an active ingredient, exhibits the effect of preventing or treating Th2-mediated immune disease. More specifically, the composition of the present invention exhibits the effect of preventing or alleviating allergic disease. Even more specifically, the composition of the present invention exhibits the effect of preventing or alleviating atopic skin disease.

The pharmaceutical composition for preventing or treating Th1 or Th2-mediated immune disease according to the present invention contains, in addition to 4H3MC (4-hydroxy-3-methoxycinnamaldehyde) as an active ingredient, a pharmaceutically acceptable carrier.

Examples of the pharmaceutically acceptable carrier that is comprised in the pharmaceutical composition of the present invention include, but are not limited to, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxylbenzoate, talc, magnesium stearate, and mineral oil, which are generally used for formulation. The pharmaceutical composition of the present invention may further comprise, in addition to the above-described components, a lubricant, a wetting agent, a sweetening agent, a flavoring agent, an emulsifier, a suspending agent, a preservative, etc. Suitable pharmaceutically acceptable carriers and formulations are described in detail in Remington's Pharmaceutical Sciences (19th ed., 1995).

The present invention is directed to a method of preventing or treating Th1 or Th2-mediated immune disease, the method comprising a step of administering 4H3MC (4-hydroxy-3-methoxycinnamaldehyde) to a subject. Herein, the 4H3MC (4-hydroxy-3-methoxycinnamaldehyde) may be in the form of a composition comprising the 4H3MC (4-hydroxy-3-methoxycinnamaldehyde). The subject may be a subject diagnosed of having Th1 or Th2-mediated immune disease, or a subject suffering from the disease, or a subject who is highly likely to suffer from the disease.

The pharmaceutical composition of the present invention is preferably administered orally. The suitable dose of the pharmaceutical composition of the present invention may vary depending on various factors, including a method for formulation, the mode of administration, the patient's age, body weight, sex, the severity of the disease, diet, the time of administration, the route of administration, excretion rate, and reaction sensitivity, and an ordinarily skilled practitioner can easily determine and prescribe the dose effective for the desired treatment. Meanwhile, the dose of the pharmaceutical composition of the present invention is preferably 0.001-2000 mg/kg (body weight) per day.

The pharmaceutical composition of the present invention may be formulated using a pharmaceutically acceptable carrier and/or excipient according to a method that can be easily implemented by a person skilled in the art to which the present invention pertains. The pharmaceutical composition of the present invention may be prepared in unit dosage form or contained in multi-dosage containers. Herein, the formulation may be in the form of solution, suspension or emulsion in an oily or aqueous vehicle, or in the form of extract, powder, granule, tablet or capsule, and may further comprising a dispersing agent or a stabilizing agent.

The present invention is also directed to a method for preventing or alleviating Th1- or Th2-mediated immune disease, the method comprising a step of administering 4H3MC (4-hydroxy-3-methoxycinnamaldehyde) to a subject. Herein, the 4H3MC (4-hydroxy-3-methoxycinnamaldehyde) may be in the form of a food composition comprising the 4H3MC (4-hydroxy-3-methoxycinnamaldehyde). Therefore, the present invention may be directed to a method for preventing or alleviating Th1- or Th2-mediated immune disease, the method comprising a step of administering to a subject a food composition comprising 4H3MC (4-hydroxy-3-methoxycinnamaldehyde).

The composition for preventing or alleviating Th1- or Th2-mediated immune disease according to the present invention may be prepared as a food, particularly a functional food composition. According to one embodiment of the present invention, 4H3MC that is used in the present invention may be one isolated from a Curcuma longa extract. Thus, the functional food composition may comprise, as an active ingredient, 4H3MC contained in a Curcuma longa extract.

The functional food composition of the present invention comprises components that are generally added for food preparation. For example, it comprises proteins, carbohydrates, fats, nutrients and seasonings. For example, when the functional food composition of the present invention is prepared as a drink, it may further comprise, in addition to 4H3MC as an active ingredient, a flavoring agent or a natural carbohydrate. Examples of the natural carbohydrate include monosaccharides (e.g., glucose, fructose, etc.), disaccharides (e.g., maltose, sucrose, etc.), oligosaccharides, polysaccharides (e.g., dextrin, cyclodextrin, etc.), and sugar alcohols (e.g., xylitol, sorbitol, erythritol, etc.). Examples of the flavoring agent that is used in the present invention include natural flavorings (e.g., thaumatin, stevia extracts, etc.) and synthetic flavorings (e.g., saccharin, aspartame, etc.). Because of easy access to food, the food composition of the present invention is very useful for the prevention or alleviation of atopic disease and Th2-mediated immune disease.

The present invention is also directed to a method for preventing or alleviating Th1- or Th2-mediated immune disease, the method comprising applying 4H3MC (4-hydroxy-3-methoxycinnamaldehyde) to the skin. Herein, the 4H3MC (4-hydroxy-3-methoxycinnamaldehyde) may be in the form of a cosmetic composition comprising the 4H3MC (4-hydroxy-3-methoxycinnamaldehyde). Therefore, the present invention may be directed to a method for preventing or alleviating Th1- or Th2-mediated immune disease, the method comprising applying to the skin a cosmetic composition comprising 4H3MC (4-hydroxy-3-methoxycinnamaldehyde).

The composition for preventing or alleviating Th1- or Th2-mediated immune disease according to the present invention may be prepared as a cosmetic composition. According to one embodiment of the present invention, 4H3MC that is used in the present invention may be one isolated from a Curcuma longa extract. Thus, the cosmetic composition may comprise, as an active ingredient, 4H3MC contained in a Curcuma longa extract. Where the composition for preventing or alleviating Th1- or Th2-mediated immune disease according to the present invention, which comprises 4H3MC as an active ingredient, is prepared as a cosmetic composition, it comprises, in addition to 4H3MC as an active ingredient, components that are generally used in cosmetic compositions. For example, it comprises conventional additives, such as a stabilizer, a solubilizer, vitamin, a pigment and fragrance, and a carrier.

The cosmetic composition of the present invention may be prepared as any formulation that is generally prepared in the art to which the present invention pertains. For example, it may be formulated as solutions, suspensions, emulsions, pastes, gels, cream, lotion, powders, soap, surfactant-containing cleansing oils, powdery foundations, emulsion foundations, wax foundations, and spray formulations, but is not limited thereto. More specifically, it may be formulated as a skin softener, milk lotion, nourishing cream, massage cream, essence, eye cream, cleansing cream, cleansing foam, cleansing water, pack, spray or powder formulation.

When the formulation of the present invention is a paste, cream or gel formulation, the carrier component used in the formulation may be animal oil, vegetable oil, wax, paraffin, starch, tragacanth, a cellulose derivative, polyethylene glycol, silicone, bentonite, silica, talc or zinc oxide, etc.

When the formulation of the present invention is a powder or spray formulation, the carrier component used in the formulation may be lactose, talc, silica, aluminum hydroxide, calcium silicate, or polyamide powder. Particularly, when the formulation is a spray formulation, it may further comprise a propellant such as chlorofluorohydrocarbon, propane/butane or dimethyl ether.

When the formulation of the present invention is a solution or emulsion formulation, the carrier component used in the formulation may be a solvent, a solubilizer or an emulsifier. For example, the carrier component may be water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or sorbitan fatty ester.

When the formulation of the present invention is a suspension formulation, the carrier component used in the formulation may be a liquid diluent such as water, ethanol or propylene glycol, a suspending agent such as ethoxylated isostearyl alcohol, polyoxyethylene sorbitol ester or polyoxyethylene sorbitan ester, microcrystalline cellulose, aluminum metahydroxide, bentonite, agar or tragacanth.

When the formulation of the present invention is a surfactant-containing cleansing formulation, the carrier component used in the formulation may be aliphatic alcohol sulfate, aliphatic alcohol ether sulfate, sulfosuccinic acid monoester, isethionate, an imidazolium derivative, methyl taurate, sarcosinate, fatty acid amide ether sulfate, alkylamidobetaine, aliphatic alcohol, fatty acid glyceride, fatty acid diethanolamide, vegetable oil, a lanolin derivative, or ethoxylated glycerol fatty acid ester.

The cosmetic composition of the present invention, which comprises 4H3MC as an active ingredient, specifically shows the effect of preventing or alleviating Th2-mediated disease. More specifically, it shows the effect of preventing or alleviating allergic disease. Even more specifically, it shows the effect of preventing or alleviating atopic skin disease.

### BRIEF DESCRIPTION OF THE DRAWINGS

FIG. 1 shows the structure of 4H3MC (4-hydroxy-3-methoxycinnamaldehyde).

FIG. 2A shows graphs indicating that 4H3MC inhibits IL-2 production in activated T cells. FIG. 2A shows the results obtained by pretreating Jurkat T cells with various concentrations of 4H3MC (0-250 μM) for 1 hour, and then treating the cells with anti-CD3/CD28 antibody (10 μg/ml) or PMA (100 nM)/A23187 (1 μM) for 6 hours, followed by analysis of IL-2 mRNA levels in the cells. FIG. 2B shows graphs indicating that 4H3MC inhibits IL-2 production in activated T cells. FIG. 2B shows the results obtained by pretreating human PBLs with various concentrations of 4H3MC (0-250 μM) for 1 hour, and then treating the cells with PMA (100 nM)/A23187 (1 μM) for 6 hours, followed by analysis of IL-2 mRNA levels in the cells. FIG. 2C shows graphs indicating that 4H3MC inhibits IL-2 production in activated T cells. FIG. 2C shows the results obtained by pretreating Jurkat T cells with 4H3MC (10 μM) for 1 hour, and then treating the cells with anti-CD3/CD28 antibody (10 μg/ml) or PMA (100 nM)/A23187 (1 μM) for various times, followed by analysis of IL-2 mRNA levels in the cells. FIG. 2D shows graphs indicating that 4H3MC inhibits IL-2 production in activated T cells. FIG. 2D shows the results obtained by pretreating human PBLs with 4H3MC (10 μM) for 1 hour, and then treating the cells with PMA (100 nM)/A23187 (1 μM), and then analyzing the time-dependent secretion of IL-2 from the cells.

FIGS. 3A and 3B show the results indicating that 4H3MC does not induce T-cell death at its effective concentration. Jurkat T cells were treated with various concentrations of 4H3MC. FIG. 3A shows the results of observing cells with a microscope (40×) after 16 hours of treatment with 4H3MC. Cells were stained with 7-AAD, and PE-labeled annexin V and cell death were measured by flow cytometry. FIG. 3B shows the results of analyzing cell death by staining with Hoechst dye and Phalloidin-TRITC after 16 hours of treatment.

FIG. 4A and FIG. 4B show graphs indicating that 4H3MC more effectively inhibits IL-2 production in activated T cells compared to HCA. In FIG. 4A Jurkat T cells were treated with various concentrations of 4H3MC or HCA (0-50 μM), and then the cells were stimulated with PMA (100 nM)/A23187 (1 μM). After 36 hours, the supernatants were collected, and IL-2 secretion was analyzed by ELISA. In FIG. 4B, human PBLs (B) were treated with various concentrations of 4H3MC or HCA (0-50 μM), and then the cells were stimulated with PMA (100 nM)/A23187 (1 μM). After 36 hours, the supernatants were collected, and IL-2 secretion was analyzed by ELISA.

FIG. 5A shows the results of analyzing the docking of 4H3MC and HCA into active sites of PKC isotypes in comparison with previously reported ligands by use of GOLD Suite v5.2. FIG. 5B shows the results of analyzing the docking of 4H3MC and HCA into active sites of PKC isotypes in comparison with previously reported ligands by use of GOLD Suite v5.2. FIG. 5C shows the results of analyzing the docking of 4H3MC and HCA into active sites of PKC isotypes in comparison with previously reported ligands by use of GOLD Suite v5.2.

FIG. 6A and FIG. 6B show graphs indicating that 4H3MC and HCA inhibit PKC activity in vitro. In FIG. 6A, Jurkat T cells were lysed, and the cytosol fraction was treated with various concentrations of 4H3MC (0.01, 0.05, 0.1, 1, 10 and 50 μM), and then treated with PMA (100 nM) for 5 minutes, followed by analysis of PKC activity. STSN (10 nM) was used as a positive control. In FIG. 6B, Jurkat T cells were lysed, and the cytosol fraction was treated with various concentrations of HCA (0.01, 0.05, 0.1, 1, 10 and 50 μM), and then treated with PMA (100 nM) for 5 minutes, followed by analysis of PKC activity. STSN (10 nM) was used as a positive control.

FIG. 7A and FIG. 7B show that 4H3MC inhibits PKCθ and MAP kinase pathways in activated T cells. In FIG. 7A, SEE-pulsed Raji B cells stained with CMRA were mixed with the same number of Jurkat T cells treated with 4H3MC (10 μM), and then the cells were incubated for 1 hour. The conjugates were fixed, immunostained with anti p-PKCθ and t-PKCθ antibodies, and then analyzed with a confocal microscope. The distribution of fluorescent signals was enumerated by FLOWVIEW. The translocation of p-PKCθ or t-PKCθ to the contact sites of T-cell-B-cell was quantified. In FIG. 7B, Jurkat T cells were pretreated with 4H3MC (10 μM) for 1 hour, and then stimulated with anti-CD3/CD28 or PMA/A23187. At the indicated time points, total (t) or phospho (p) forms of Zap70, PKCθ, ERK and p38 kinase were analyzed by Western blot.

FIG. 8 shows that 4H3MC inhibits AP-1, NF-κB and NFAT pathways in activated T cells. Jurkat T cells were transfected for 24 hours with pGL3-AP-1, NF-κB and NFAT vector plasmids with pRLTK. The cells were pretreated with 4H3MC (10 μM) for 1 hour, and then stimulated with PMA/A23187 for 16 hours. The luciferase activities were analyzed by a luminometer.

FIG. 9A and FIG. 9B show that 4H3MC inhibits IL-2 production and the activation of PKCθ and MAP in pre-activated T cells. In FIG. 9A, Jurkat T cells were stimulated with anti-CD3/CD28 antibody or PMA/A23187 for 30 minutes, and then treated with 4H3MC (10 μM). IL-2 mRNA levels were analyzed at the indicated time intervals. In FIG. 9B, at the indicated time points, the samples of (A) were analyzed by Western blot analysis.

FIG. 10A, FIG. 10B and FIG. 10C show that, one month after oral administration of 4H3MC to mice with induced atopic disease, the ear thickness was reduced and inflammatory reactions were alleviated. In FIG. 10A, when DNCB and mite were alternately applied to the ear twice a week, atopic disease was induced after 28 days. 1 mg of 4H3MC was administered orally to each mouse once a day, and the ear thickness was measured twice a week to confirm induction of atopic disease. In FIG. 10B, the degree of induction of atopy was quantified by measuring the ear thickness of each group twice a week for 28 days. FIG. 10C shows photographs of the ears of each group, measured after 28 days.

### DESCRIPTION OF SPECIFIC EMBODIMENTS

Hereinafter, the present invention will be described in further detail with reference to examples. It will be obvious those skilled in the art that these examples are for illustrative purposes only and are not intended to limit the scope of the present invention.

### EXAMPLES

Materials and Methods

Cell Culture

Jurkat T cells (ATCC, CRL-1651, Manassas, Va.) and Raji B cells (ATCC, CCL-86) were cultured in RPMI complete medium supplemented with 10% fetal bovine serum (FBS, AusGeneX, Santa Clara, Calif.) and PenStrep (Gibco-RBL). After written informed consent, human peripheral blood leukocytes (PBL) were isolated from healthy donors by a sedimentation method using dextran and Ficoll Amersham Biosciences, Piscataway, N.J.). All the cell lines and cells used in the present invention were cultured at 37° C. in a humidified incubator containing 5% CO₂. All experiments using human peripheral blood leukocytes were approved by the Ethics Committee of the School of Life Sciences, GIST.

Reagents and Antibodies

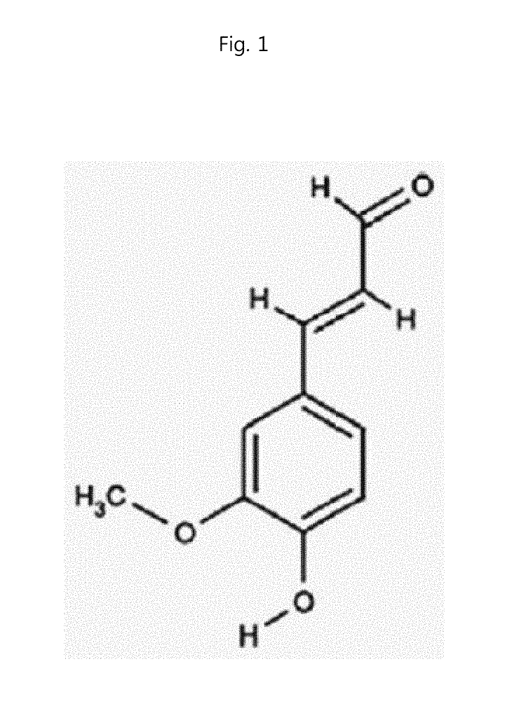

4-Hydroxy-3-methoxycinnamaldehyde (4H3MC; PubChem CID: 5280536) and p-hydroxycinnamic acid (HCA; PubChem CID: 637542) with the purity over 99% were provided by Professor Seung-Ho Lee from Yeungnam University (Korea). For treatment in the cells, 4H3MC and HCA were dissolved in DMSO, and 0.1% DMSO was used as a vehicle control. The chemical structure and properties of 4H3MC are shown in FIG. 1. Human CD3 (OKT3) and LFA-1 (TS2/4) antibodies were isolated from hybridoma cells (ATCC, HB-202, CRL-8001), anti-human CD28 antibody was purchased from R&D Systems (Minneapolis, Minn.). PMA (Phorbolmyristate actate), A23187, STSN (Staurosporine), PLL (poly-L-lysine) and TRITC-phalloidin were purchased from Sigma (St. Louis, Mo.). SEE (Staphylococcus enterotoxin) was purchased from Toxin Technology (Sarasota, Fla.). Annexin V-PE and 7-AAD was purchased from BD Biosciences (San Diego, Calif.). Cell Tracker™ Green CMFDA and Orange CMRA, Hoechst dye, cy3-conjugated goat anti-mouse antibody and Texas red conjugated goat anti-rabbit antibody were purchased from Invitrogen (Carlsbad, Calif.). Rabbit polyclonal antibodies against p-Zap70, p-PKCθ, p-ERK and ERK, p-P38 and P38, horseradish peroxidase-conjugated anti-mouse IgG and anti-rabbit IgG were purchased from Cell Signalling Technology (Beverly, Mass.). Rabbit anti-human PKCθ antibody was purchased from Santa Cruz Biotechnology (Santa Cruz, Calif.). Mouse anti-human Zap70 antibody was obtained from Merck Millipore (Germany).

T-Cell Stimulation and Treatment with 4H3MC or HCA

Jurkat T cells (5×10⁵) or human PBLs (1×10⁶) were stimulated with anti-CD3 (OKT3, 10 μg/ml)/CD28 (2 μg/ml) or PMA (100 nM)/A23187 (1 μM). For anti-CD3/CD28 stimulation, cells were placed on a culture dish coated with anti-CD3 antibody, and were then treated with anti-CD28 (2 μg/ml) antibody. For superantigen stimulation, T cells were incubated with SEE (1 μg/ml)-pulsed Raji B cells. For pre- or post-treatment experiments, various concentrations of 4H3MC or HCA were added 30 minutes or 60 minutes before stimulation.

Real-Time PCR and Digitization of Real Time-PCR Result

Total RNA was isolated from Jurkat T cells using TRIZOL reagent (JBI, Korea). Reverse transcription of the RNA was performed using RT Pre-Mix (Intron, Korea). The primers and PCR conditions were as follows:

Human IL2:
(SEQ.ID.No: 1)
5′-CACGTCTTGCAC TTGTCAC-3′
and
(SEQ.ID.No: 2)
5′-CCTTCTTGGGCATGTAAAACT-3′;
human GAPDH:
(SEQ.ID.No: 3)
5′-CGGAGTCAACGGATTTGGTCGTAT-3′
and
(SEQ.ID.No: 4)
5′-AGCCTTCTCCATGGTGGTGAAGAC-3′.

The amplification profile was composed of denaturation at 94° C. for 30 seconds, annealing at 60° C. for 20 seconds, and extension at 72° C. for 40 seconds. 30 cycles were preceded by denaturation at 72° C. for 7 minutes. In some experiments, the expression levels of IL-2 mRNA were evaluated by real-time RT-PCR. Total RNA was isolated and cDNA was synthesized as described above. PCR amplification was performed in Step One Real-time PCR system (Applied Biosystems, Foster city, CA) for continuous fluorescence detection system using SYBR (Takara, Japan). Each PCR reaction was performed under the following conditions: 94° C. for 30 sec, 60° C. for 30 sec and 72° C. for 30 sec, plate read (detection of fluorescent product) for cycles, followed by extension at 72° C. for 7 min. Melting curve analysis was done to characterize the double-stranded DNA product. The levels of IL-2 mRNA normalized for GAPDH were expressed as fold changes relative to that of untreated controls. All experiments were performed at least three times.

ELISA Assay

Jurkat T cells or human PBLs were stimulated in the same manner as described above with respect to T-cell stimulation and treatment with 4H3MC or HCA. At the indicated time points, the supernatants were collected, and the concentrations of IL-2 in the supernatants were measured using a Duo set Human IL-2 ELISA kit (R&D Systems).

Cell Death Assay Using 7-AAD and Annexin V

Cell death of Jurkat T cells was examined using a double staining method with 7-AAD and annexin V-PE. Jurkat T cells (1×10⁶) were indicated concentrations of 4H3MC for 16 hours, and then suspended in 200 μl of HBSS buffer comprising 7-AAD (1 μg/ml) and were incubated at 37° C. for 10 minutes. After 10 minutes, the cells were incubated with 200 μl of HBSS buffer comprising V-PE (20 μg/ml) and analyzed immediately on a BD FACS Canto™ II Flow Cytometer (BD Biosciences). All experiments were performed at least three times.

Hoechst Staining

Jurkat T cells were treated with 10 μM of 4H3MC for 16 hours, and then pelleted onto a PLL-coated cover glass slide (18-mm diameter; Fisher Scientific, Pittsburgh, Pa.) using a cytospin centrifuge. The pelleted cells were fixed in 2% para-formaldehyde at room temperature for 10 minutes, and then washed three times with PBS. As control, cells were stained with TRITC-phalloidin for detecting actin. The slide was incubated for 2 minutes in PBS containing Hoechst dye (1:105) at room temperature, and then the cells were examined using an FV1000 confocal microscope.

Protein Expression Analysis (Western Blotting)

Jurkat T cells were lysed by cold lysis buffer (1% Triton X-100, 150 mM NaCl, 20 mM Tris, pH 7.5, protease inhibitor and phosphatase inhibitor) for 1 hour, and then centrifuged at 4° C. at 16,000×g for 30 minutes. About 100 μg of the extract lysate was separated through 10% SDS-PAGE. Proteins were transferred into a nitrocellulose membrane by means of Trans-Blot SD semidry transfer cell (Bio-Rad, Hercules, Calif.). The membrane was blocked in 5% skim milk for 1 hour, washed, and then incubated with the indicated antibodies in 3% skim milk overnight. Excess primary antibody was removed by washing the membrane with TBST, and then the membrane was incubated with peroxidase-conjugated secondary antibody for 2 hours. After washing with TBST, bands were visualized by Western blotting detection reagent and then exposed to x-ray film.

Immunofluorescence Staining and Confocal Imaging Analysis

Jurkat T cells were pretreated with 10 μM of 4H3MC for 1 hour, while Raji B cells were stained with 1 μM of CMFDA green tracker for 30 minutes. After washing twice with RPMI medium, Raji B cells were incubated with SEE (1 μg/ml) at 37° C. for 30 minutes. SEE-conjugated Raji B cells were incubated with 4H3MC-treated Jurkat T cells for 10 minutes to form an immunological synapse, and then placed on glass coverslips. The cells were fixed with 2% paraformaldehyde and washed with PBS. After washing, the cells were permeabilized with 0.1% TritonX-100 and incubated with primary antibodies (antibodies against CD3, LFA-1, t-PKCθ, p-PKCθ) overnight. On the next day, the cells were washed with PBS, and then incubated with secondary antibodies (cy3-conjugated goat anti-mouse IgG, Texas Red-conjugated goat anti-rabbit IgG antibodies) at room temperature for 1 hour. After washing, the cells were placed on a slide glass using Dako fluorescent mounting medium (Dako, Denmark), followed by drying. The dried slide samples were examined with an FV1000 confocal laser scanning microscope (Olympus), and CD3, LFA-1, t-PKCθ and p-PKCθ accumulation at the immunological synapse was observed and analyzed.

Quantitation of T Cell-Antigen Presenting Cell Conjugates

Jurkat T cells were treated with 10 μM of 4H3MC at 37° C. for 1 hour. Then, Jurkat T cells and Raji B cells were stained with Cell Tracker Green CMFDA and Orange CMRA (Molecular Probes). Raji B cells were incubated with 1 μg/ml SEE for 30 minutes, and then suspended in RPMI medium. For conjugation, an equal number (1×10⁶) of B cells and T cells were mixed and incubated at 37° C. for 30 minutes. The relative proportion of green, orange and orange-green events in each tube was determined by BD FACS Canto™ II Flow Cytometer (BD Biosciences). The number of gated events counted per sample was at least 10,000.

AP-1, NF-κB and NFAT Luciferase Activity Assays

Jurkat T cells (1.5×10⁶) were transfected with 100 μl of Amaxa's Nucleofector solution (Amaxa, Germany) containing 3 μg of pGL3-AP-1, pGL3-NF-κB or pGL3-NFAT Luc plasmids, and then the cells were transferred to complete medium and cultured at 37° C. After 48 hours of transfection, the transfected cells were pretreated with 4H3MC (10 μM) for 1 hour, and then stimulated with PMA/A23187 for 16 hours. After 12 hours, the cells were harvested and lysed with a lysis buffer for luciferase assay (Promega, Madison, Wis.). Cellular debris was removed from lysed proteins by centrifugation at 16,000×g at 4° C. for 30 minutes. Luciferase activity was measured with a Centro LB 960 Luminometer (Berthold Technologies, Germany).

PKC Activity Measurement

Jurkat T cells (1×10⁶) were suspended in lysis buffer (1% Triton X-100, 150 mM NaCl, 20 mM Tris pH 7.5, protease inhibitor, phosphatase inhibitor), and then kept at 4° C. for 1 hour and centrifuged at 14,000×g for 30 minutes. Cell lysate was incubated with 4H3MC (0.0150 μM), HCA (0.0150 μM) or STSN (10 nM) at 4° C. for 30 minutes. PMA (100 nM) was added, and PKC activity was measured with a nonradioactive protein kinase assay based on ELISA. The assay was developed with tetramethylbenzidine substrate, and the color developed proportionally to PKC phosphotransferase activity. The intensity of the color was measured at 450 nm. The data were expressed as relative kinase activity.

Docking Studies

4H3MC and HCA were docked using GOLD Suite v5.2 (The Cambridge Crystallographic Data Centre Inc., New Jersey). Low-energy conformers of the two compounds were generated by MarvinSketch version 6.1.3 (ChemAxon, Hungary) and then docked by GOLD Suite v5.2 into the binding cavity present in PKC isotypes using ChemPLP scoring system. About fifty poses were kept for each conformer. These poses were then recorded by GOLD Suite v5.2 using GoldScore and ChemScore scoring systems. Visual inspection was carried out by Hermes interface to GOLD Suite v5.2, and Discovery Studio 4.0 Client (Accelrys, Inc., CA). SuperPred Target Prediction Server was used for target prediction of 4H3MC.

Experiment on the Effect of 4H3MC on Treatment of Atopic Skin Disease in Mice

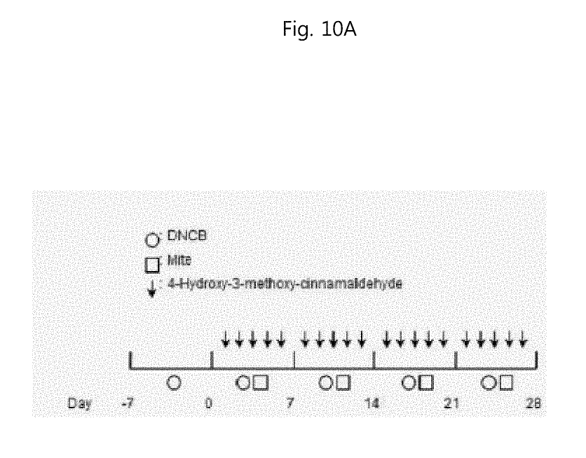

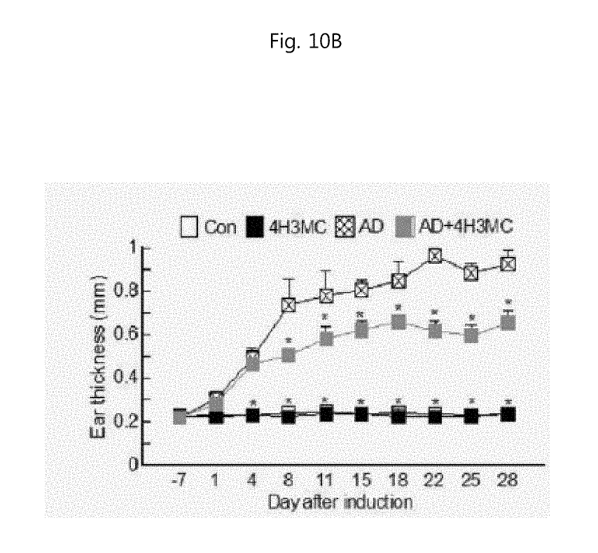

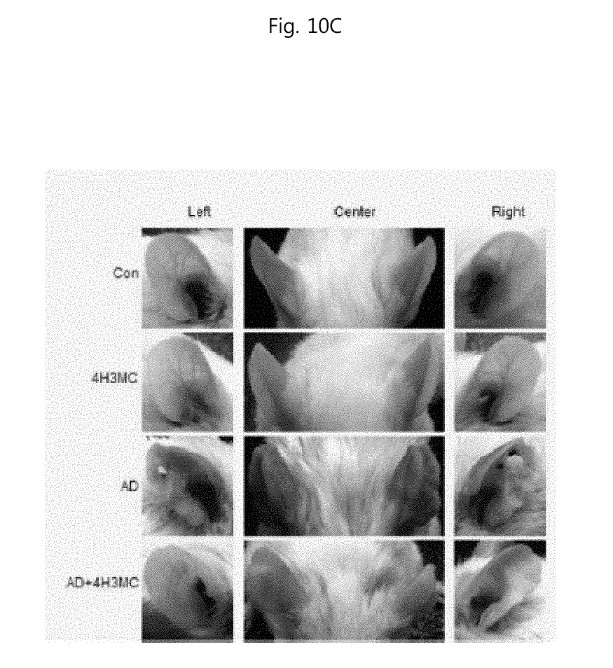

With reference to a previously reported publication, atopic skin disease was induced using DNCB and a mite extract [14]. A method for carrying out the experiment is shown in FIG. 10A. BALB/c mice were divided into four groups, and then the skin was peeled off by touching each earlobe five times with a surgical tape (Seo-il Chemistry, Hwa-sung, Korea). On day 0, 20 μl of DNCB (1%) was applied to each peeled earlobe, and on day 4, 20 μl of a mite extract (10 mg/mL) was applied. DNCB and the mite extract were alternately applied once a week for 4 weeks. From day 1, 4H3MC (50 mg/kg) was administered orally everyday for 4 weeks. Using a thickness meter (Kori Seiki MFG Co., Japan), the ear thickness was measured at 24 hours after application of DNCB or the mite extract. After 28 days, blood was sampled, and the serum portions were stored at 70° C. After blood sampling, the ear tissue was cut and used for histopathological analysis.

### Experimental Results

4H3MC Inhibits IL-2 Production in Activated T Cells

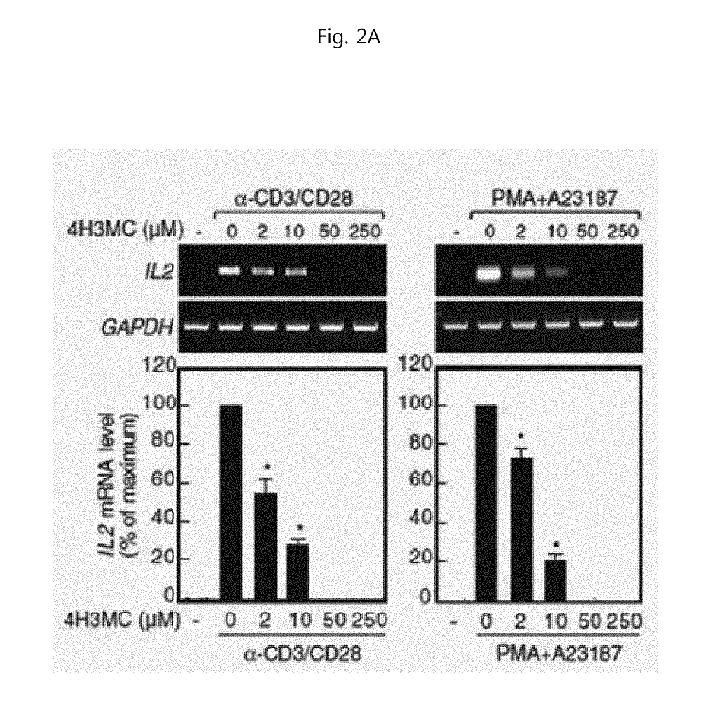

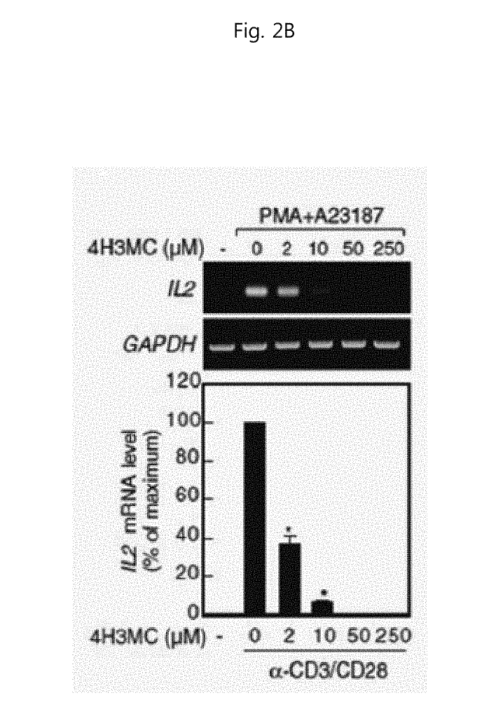

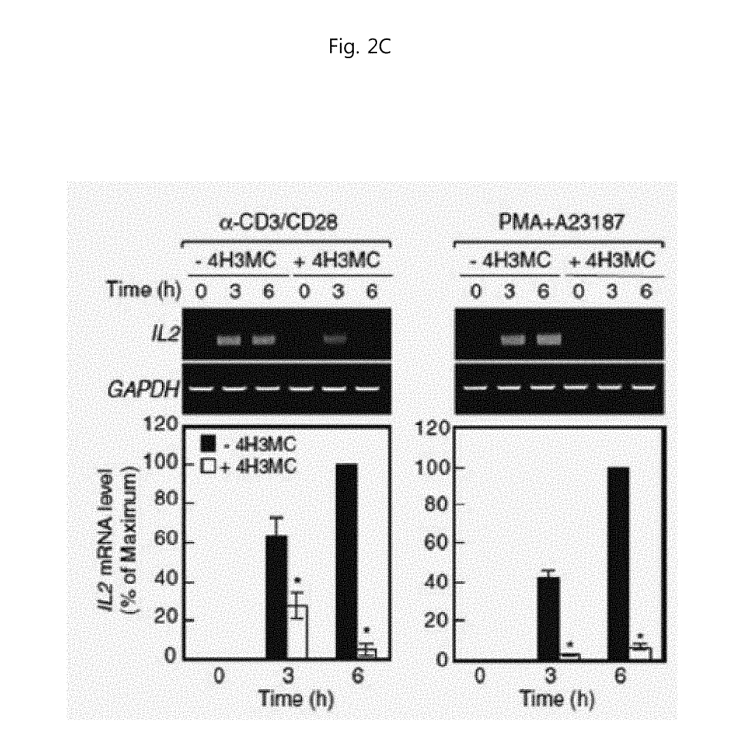

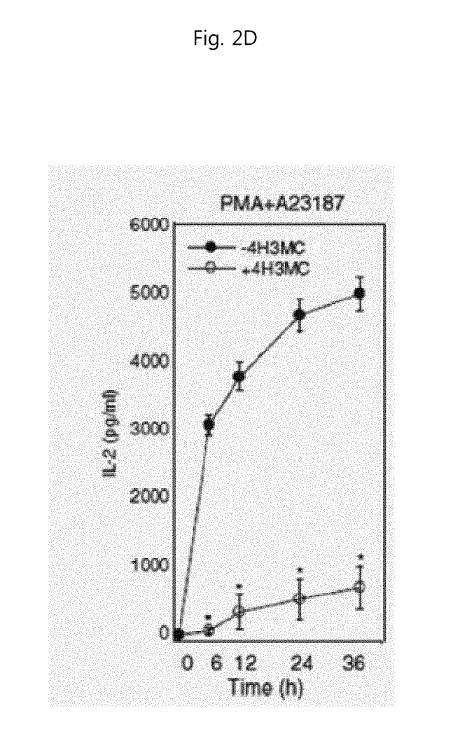

Whether 4H3MC can inhibit T-cell activation was examined. As IL-2 is produced and released upon T-cell activation, the effect of 4H3MC on IL-2 secretion from T cells was examined. Jurkat T cells (FIG. 2A) and human PBLs (FIG. 2B) were pretreated with various concentrations of 4H3MC for 1 hour, and then treated with PMA/A23187 or anti-CD3/CD28 antibody. As a result, 4H3MC significantly inhibited IL-2 expression in a concentration-dependent manner (FIGS. 2A and 2B). Particularly, dramatic inhibition could be seen at a concentration of 2-10 μM. Time-dependent experiments revealed that 4H3MC inhibited IL-2 mRNA expression at the early time point (about 3 hours) after stimulation which lasted over 6 hours (FIG. 2C). Similar to gene expression in Jurkat T cells, IL-2 secretion was significantly reduced in 4H3MC-treated human PBLs as well (FIG. 2D). Such results indicate that 4H3MC has the effect of inhibiting T-cell activation.

4H3MC does not Induce T-Cell Apoptosis at Effective Concentration

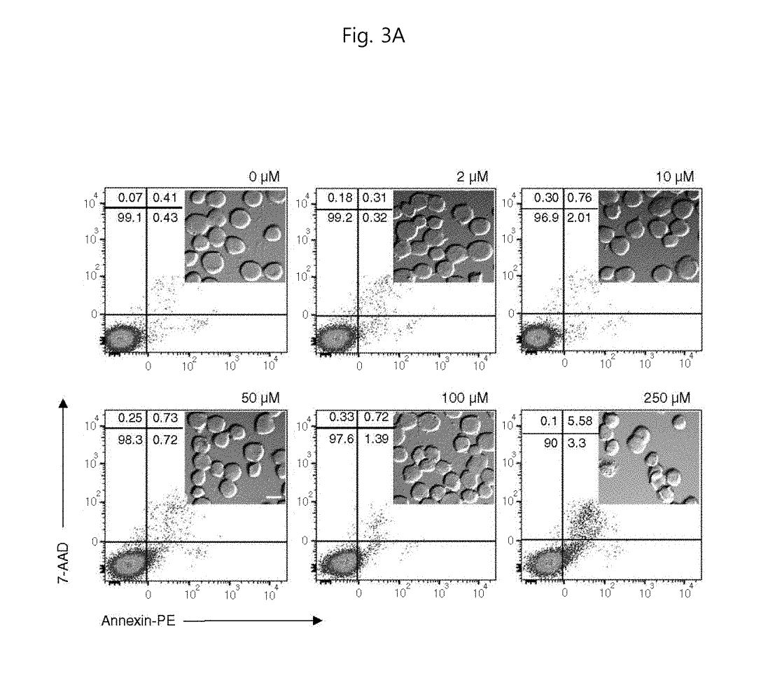

In order to demonstrate the potency and long duration of action of 4H3MC, the cytotoxicity of 4H3MC was tested. 4H3MC did not induce apoptosis or necrosis at a concentration of 10-100 μM, although high concentrations (250 μM) induced necrosis in some Jurkat T cells (FIG. 3A). A consistent result was reflected by flow cytometric analysis (FIG. 3A). In addition, Hoechst staining indicated that 4H3MC does not induce apoptosis or necrosis at its effective concentration (FIG. 3B).

4H3MC Inhibits T-Cell Activation More Effectively than HCA

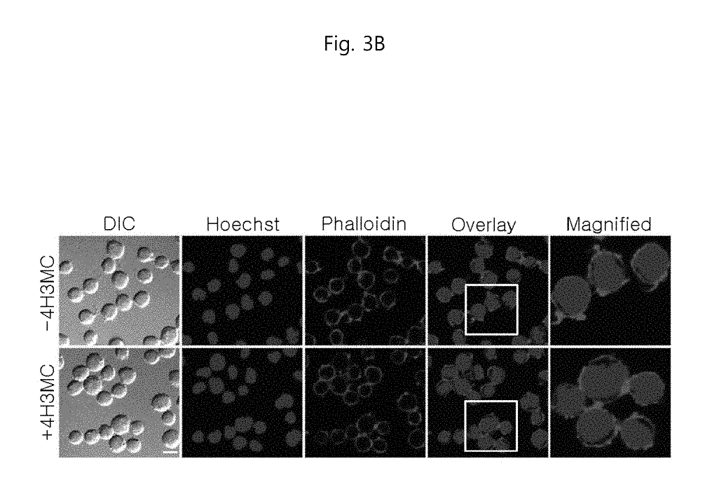

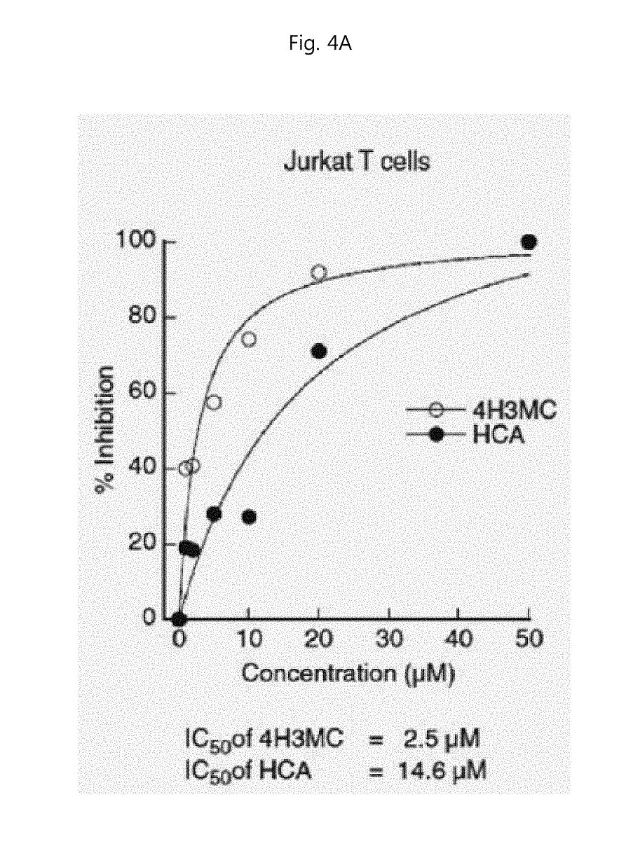

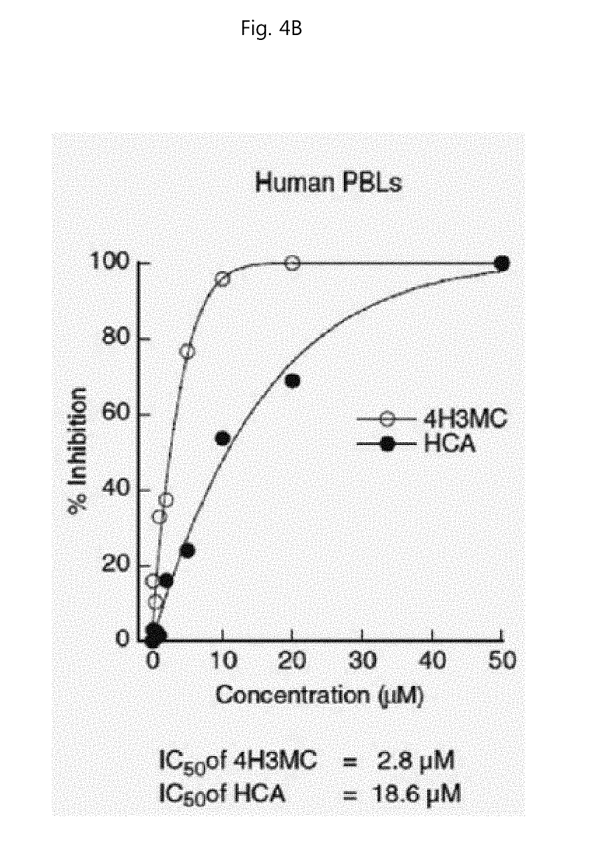

The effect of 4H3MC was verified by comparing its activity with that of HCA. To this end, Jurkat T cells and human PBLs were pretreated with 4H3MC or HCA for hour, and then stimulated with PMA/A23187. 4H3MC effectively inhibited IL-2 secretion at an IC₅₀ of about 2.5 μM, whereas HCA exerted at its effects at an IC₅₀ of about 15-18 μM (FIG. 4A and FIG. 4B). This suggests that 4H3MC is nearly 5-fold effective than HCA in the inhibition of IL-2 in T cells.

4H3MC Inhibits PKC Kinase Activity More Effectively than HCA

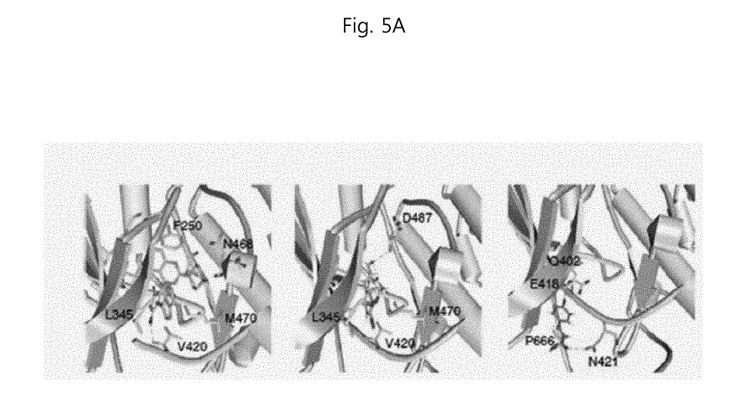

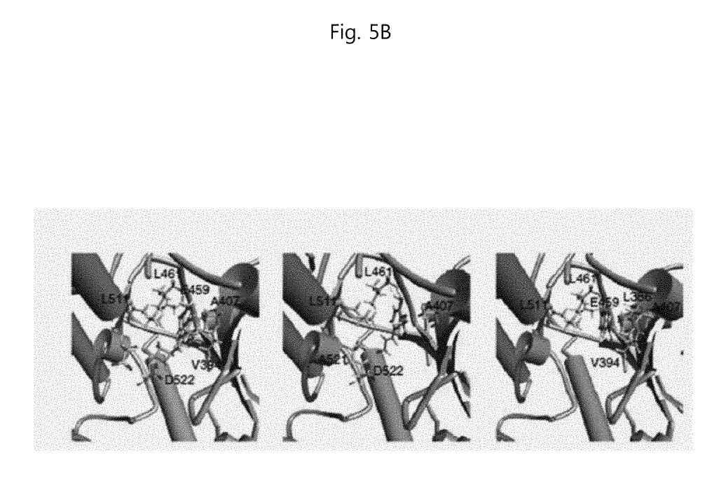

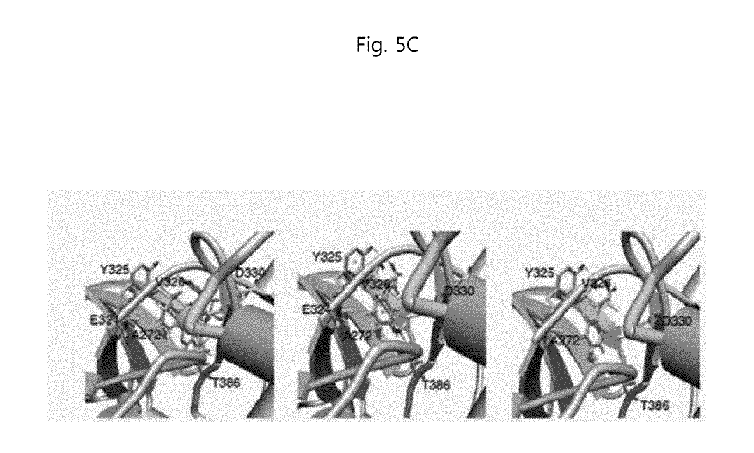

To unravel the inhibitory effect and molecular mechanism of 4H3MC as described above, it was tried to map the target of 4H3MC using SuperPred Target Prediction Server [17] for small molecular target prediction. As a result, PKCι was predicted to be a possible target. As it was previously reported that HCA inhibits PKC phosphorylation [13], the binding compatibility of 4H3MC and HCA to PKCθ was examined computationally. In addition, the present inventors performed studies by docking the low-energy conformers of 4H3MC and HCA into the binding site of PKC isotypes using GOLD Suite v5.2. Docking analysis revealed the fit of 4H3MC to the ATP-binding sites of PKC isotypes (FIG. 5A, FIG. 5B and FIG. 5C), and computational analysis indicated that 4H3MC binds to PKC isotypes more easily than HCA.

Because PKCα and PKCθ are highly expressed in T cells and PKCι was the target predicted by SuperPred, the binding poses of 4H3MC and HCA with PKCα, PKCθ and PKCι were analyzed to identify important amino acid residues involved in the binding and compared with their respective known inhibitors. 4H3MC firmly binds to the catalytic site of PKCα with three hydrogen bonds making a strong contribution to ligand-protein affinity with the highest docking score (FIG. 5A-b). HCA also binds to the same pocket with a different pose through hydrogen bonding with E418 similar to standard inhibitor AEB071 (FIG. 5A-c).

Docking poses of staurosporine, sotrastaurin and other PKC inhibitors have revealed the importance of hydrogen bond interaction with L461 of PKCθ as of utmost importance, although interactions with other amino acids of ATP-binding pocket, including V394, A407, L461, L511, D520, A521 and D522, are also important [27].

4H3MC and HCA bind to the ATP-binding pocket of PKCθ via 3 hydrogen bonds and fit well into the hydrophobic pocket composed of V394, A407, Leu461, L511, A521 and D522 utilizing several non-covalent interactions (FIG. 5B-b, c), suggesting them to be the ATP-competitive agents. In addition, 4H3MC and HCA showed good binding to the nucleotide binding site of PKCι as well with the aromatic ring of 4H3MC playing key role in the interactions (FIG. 5C-b).

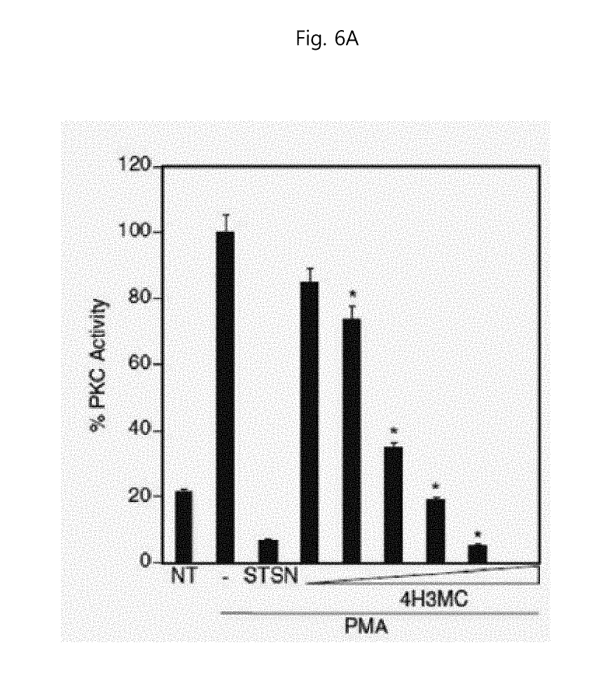

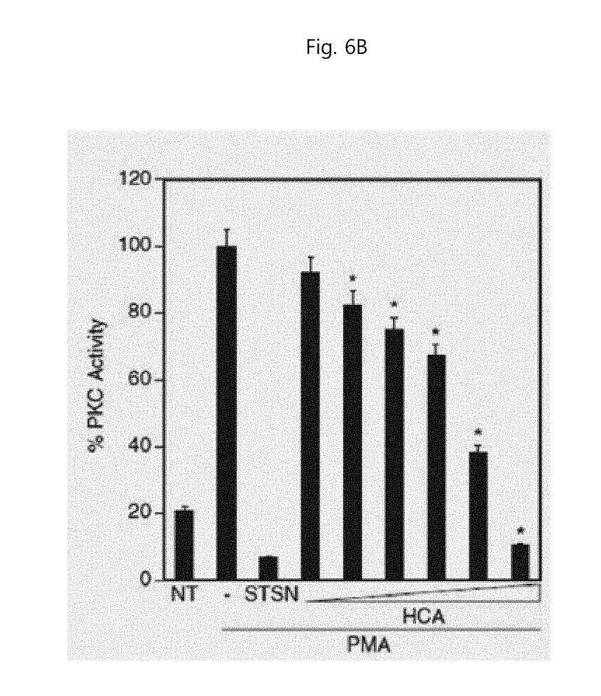

To confirm the predicted target in vitro, the effects of 4H3MC and HCA were measured by PKC activity assay. In accordance with computational analysis results, 4H3MC significantly inhibited PKC-specific activity with the lower effective concentration than HCA (FIG. 6A and FIG. 6B), indicating that 4H3MC is a better therapeutic option for immunomodulation.

4H3MC Inhibits PKCθ Phosphorylation and its Accumulation at IS

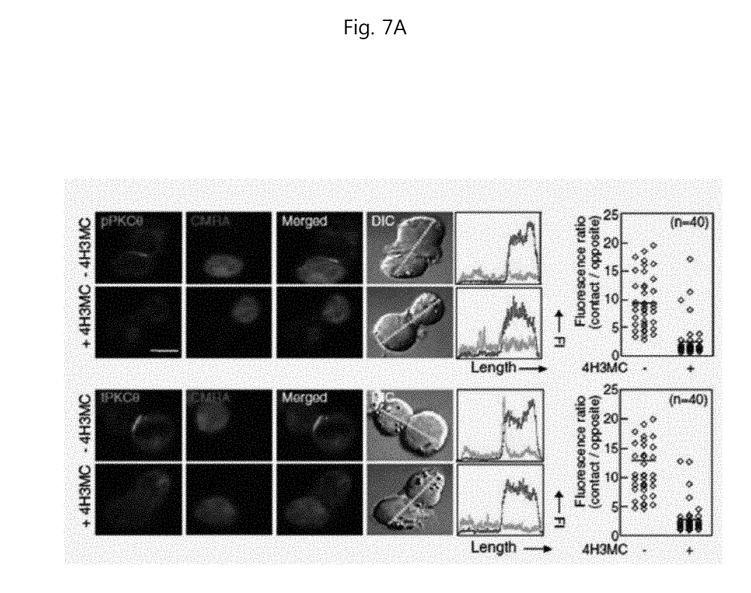

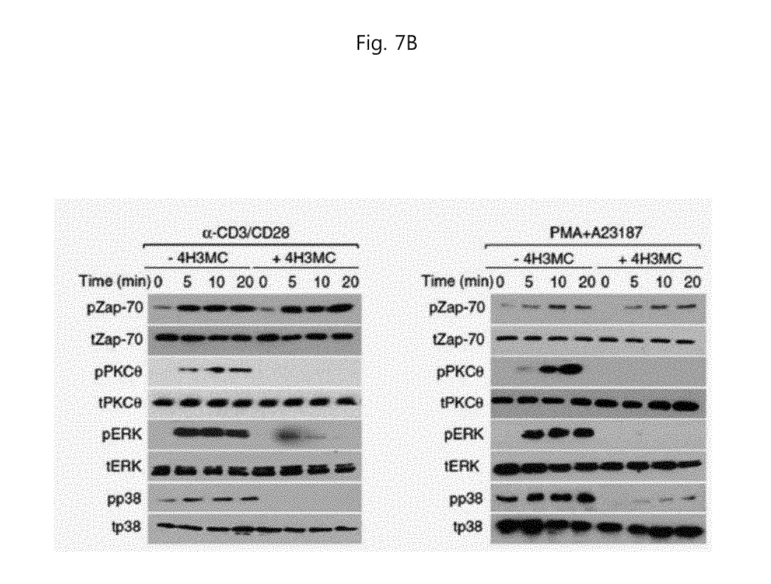

To mimic a physiologic response, Jurkat T cells were subjected to form conjugates with SEE-pulsed Raji B cells, and then the localization of phosphorylated and total forms of PKCθ was scanned by confocal microscopy (FIG. 7A). Pretreatment with 4H3MC significantly reduced the phosphorylation as well as accumulation of PKCθ at the IS. In addition, 4H3MC significantly blocked phosphorylation of PKCθ in Jurkat T cells treated with anti-CD3/CD28 or PMA/A23187 (FIG. 7B). Such results suggest that PKCθ is the eventual molecular target for 4H3MC effectiveness in T cells.

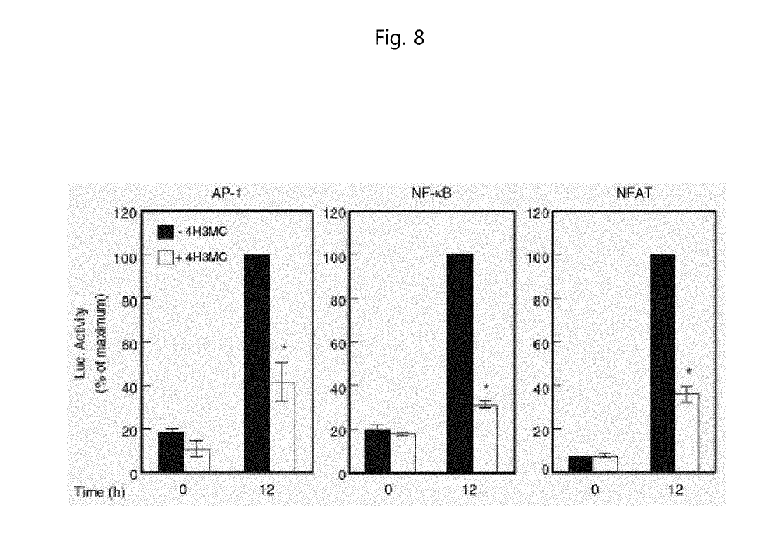

To understand the effect of 4H3MC on downstream pathways, the phosphorylation of MAP kinase was examined. Activation of MAP kinase is crucial for transcriptional and non-transcriptional responses of the immune system, playing essential roles in the development, homeostasis, proliferation, immune response signaling and apoptosis of T cells [29]. Members of MAP kinases, including p38, ERK and JNK, are central in immunological signal transduction pathways and regulate transcriptional activities of NF-κB, AP-1 and NFAT in activated T cells [30]. Different isotypes of PKC are known to be involved in MAP kinase activity. As shown in FIG. 7B, pretreatment, with 4H3MC dramatically reduced anti-CD3/CD28- and PMA/A23187-induced phosphorylation of ERK and p38. Moreover, pretreatment with 4H3MC considerably reduced PMA/A23187-induced luciferase activities of NF-κB, AP-1 and NFAT in Jurkat T cells (FIG. 8).

Post-Treatment with 4H3MC Inhibits IL-2 Production in Activated T Cells

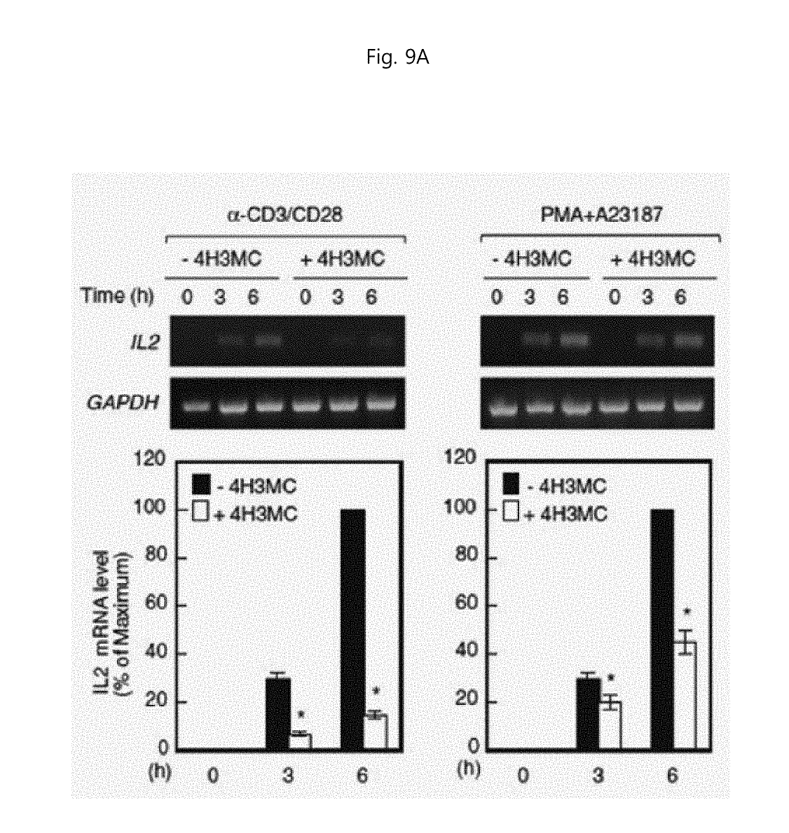

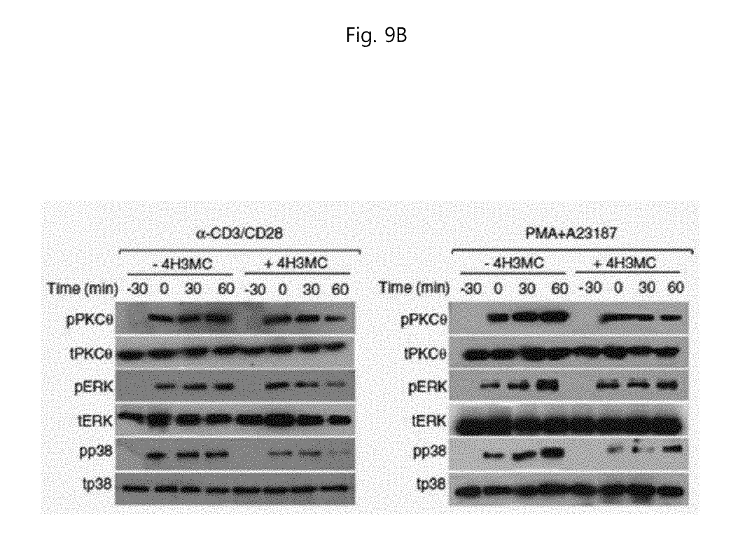

To understand whether 4H3MC has only a preventive effect or, otherwise, it also has a therapeutic effectiveness to modulate the activity of pre-activated T cells, the efficacy of 4H3MC was checked after stimulation of T cells with anti-CD3/28 or PMA+A23187. Time-dependent experiments revealed that post-treatment (therapeutic regimen) also effectively reduced the expression of IL2 mRNA in pre-activated Jurkat T cells (FIG. 9A). In addition, long-lasting phosphorylate forms of PKCθ, ERK and p38 were significantly reduced after treatment with 4H3MC (FIG. 9B).

4H3MC Shows a Therapeutic Effect Against Atopic Skin Disease

Through the examples as described above, the present inventors have found that 4H3MC is not involved in cell death and inhibit T-cell activity by inhibiting PKC signaling. Based on this fact, a test was made of whether 4H3MC can exhibit a therapeutic effect on atopic disease, the development and maintenance of which is greatly influenced by T cells. For the test, DNCB and a mite extract were applied to the ears of BALB/c mice to make atopic disease models, and 4H3MC was administered orally to the mice in a scheduled manner (FIG. 10A). The ear thickness was measured and analyzed comparatively between the animal groups, and as a result, it was shown that the ear thickness of the group with atopic disease was thicker as the disease was more severe. In the case of the atopic disease group mice administered orally with 4H3MC, it could be seen that the ear thickness became significantly thinner from 8 days (FIG. 10B). Meanwhile, 28 days after induction of the disease, the atopic disease mice showed the symptoms in which the ear was flushed and swollen, whereas in the mice administered orally with 4H3MC, such symptoms were alleviated (FIG. 10C). Such results suggest that 4H3MC can alleviate symptoms of atopic disease and can effectively modulate immune responses.

The features and advantages of the present invention are summarized as follows:

(a) The present invention provides a pharmaceutical composition for preventing or treating Th1- or Th2-mediated immune disease, which comprises 4H3MC (4-hydroxy-3-methoxycinnamaldehyde) as an active ingredient.

(b) The present invention provides a food composition or cosmetic composition for preventing or alleviating Th1- or Th2-mediated immune disease, which comprises 4H3MC (4-hydroxy-3-methoxycinnamaldehyde) as an active ingredient.

(c) 4H3MC (4-hydroxy-3-methoxycinnamaldehyde) according to the present invention exhibits the effect of inhibiting T-cell activity without inducing T-cell death, and thus can be developed into an effective therapeutic agent against Th1- or Th2-mediated immune disease.

Although the present disclosure has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereof.

### REFERENCES

[1] Dejaco C, Duftner C, Grubeck-Loebenstein B, Schirmer M. Imbalance of regulatory T cells in human autoimmune diseases. Immunology 2006; 117(3):289.300.
[2] Luppi P. How immune mechanisms are affected by pregnancy. Vaccine 2003; 21(24):3352.7.
[3] Wang H, Kadlecek T A, Au-Yeung B B, Goodfellow H E S, Hsu L-Y, Freedman T S, et al. ZAP-70: an essential kinase in T-cell signaling”. Cold Spring Harb Perspect Biol 2010; vol. 2(5):a002279.
[4] Elder M E, Lin D, Clever J, Chan A C, Hope T J, Weiss A, et al. Human severe combined immunodeficiency due to a defect in ZAP-70, a T cell tyrosine kinase. Science 1994; 264(5165):15969.
[5] Ben-Neriah Y, Karin M. Inflammation meets cancer, with NF-κB as the matchmaker. Nat Immunol 2011; 12(8):71523.
[6] Lawrence T. The nuclear factor N F-kappaB pathway in inflammation. Cold Spring Harb Perspect Biol 2009; vol. 1(6):a001651.
[7] Schmitz M L, Bacher S, Dienz 0. N F-kappaB activation pathways induced by T cell costimulation. FASEB J 2003; 17(15):2187]93.
[8] Chen H-C, Song J, Williams C M, Shuford C M, Liu J, Wang J P, et al. Monolignol pathway 4-coumaric acid:coenzyme A ligases in Populus trichocarpa: novel specificity, metabolic regulation, and simulation of coenzyme A ligation fluxes. Plant Physiol 2013; 161(3):1501116.
[9] Humphreys J M, Chapple C. Rewriting the lignin roadmap. Curr Opin Plant Biol 2002; 5(3):2249.
[10] “Influences of cinnamic aldehydes on H+ extrusion activity and ultrastructure of Candida.”
[11] Chen H-H, ChiangW, Chang J-Y, Chien Y-L, Lee C-K, Liu K-J, et al. Antimutagenic constituents of adlay (Coix lachryma-jobi L. var. ma-yuen Stapf) with potential cancer chemopreventive activity. J Agric Food Chem 2011; 59(12):6444152.
[12] Roh E, Jeong I-Y, Shin H, Song S, Doo Kim N, Jung S-H, et al. Downregulation of melanocyte-specific facultative melanogenesis by 4-hydroxy-3-methoxycinnamaldehyde acting as a cAMP antagonist. J Invest Dermatol 2014; 134(2):5513.
[13] Lee H-S, Kim Y-D, Na B-R, Kim H-R, Choi E-J, Han W-C, et al. Phytocomponent p-Hydroxycinnamic acid inhibits T-cell activation by modulation of protein kinase C-θ-dependent pathway. Int Immunopharmacol 2012; 12(1):1318.
[14] Briken V. Molecular mechanisms of host-pathogen interactions and their potential for the discovery of new drug targets. Curr Drug Targets 2008; 9(2):15047.
[15] Barh D, Tiwari S, Jain N, Ali A, Santos A R, Misra A N, et al. In silico subtractive genomics for target identification in human bacterial pathogens. Drug Dev Res 2011; 72(2):16277.
[16] Anishetty S, Pulimi M, Pennathur G. Potential drug targets in Mycobacterium tuberculosis through metabolic pathway analysis. Comput Biol Chem 2005; 29(5):368678.
[17] Dunkel M, Gunther S, Ahmed J, Wittig B, Preissner R. SuperPred: drug classification and target prediction. Nucleic Acids Res 2008; vol. 36(Web Server issue):W55t9.
[18] Spitaler M, Cantrell D A. Protein kinase C and beyond. Nat Immunol 2004; 5(8): 785590.
[19] Bauer B, Krumbock N, Ghaffari-Tabrizi N, Kampfer S, Villunger A, Wilda M, et al. T cell expressed PKCtheta demonstrates cell-type selective function. Eur J Immunol 2000; 30(12):3645054.
[20] Manicassamy S, Gupta S, Sun Z. Selective function of PKC-theta in T cells. Cell Mol Immunol 2006; 3(4):263070.
[21] Tan S-L, Zhao J, Bi C, Chen X C, Hepburn D L, Wang J, et al. Resistance to experimental autoimmune encephalomyelitis and impaired IL-17 production in protein kinase C theta-deficient mice. J Immunol 2006; 176(5):28729.
[22] Ou C-C, Hsiao Y-M, Wu W-J, Tasy G J, Ko J-L, Lin M-Y. FIP-fve stimulates interferon gamma production via modulation of calcium release and PKC-alpha activation. J Agric Food Chem 2009; 57(22):11008213.
[23] Meisel M, Hermann-Kleiter N, Hinterleitner R, Gruber T, Wachowicz K, Pfeifhofer-Obermair C, et al. The kinase PKCα selectively upregulates interleukin-17A during Th17 cell immune responses. Immunity 2013; 38(1):4152.
[24] Chaudhary D, Kasaian M. PKCtheta: a potential therapeutic target for T-cell mediated diseases. Curr Opin Investig Drugs 2006; 7(5):43247.
[25] Morgan M M, Labno C M, Van Seventer G a, Denny M F, Straus D B, Burkhardt J K. Superantigen-induced T cell:B cell conjugation is mediated by LFA-1 and requires signaling through Lck, but not ZAP-70. J Immunol 2001; vol. 167(10):5708518.
[26] Schmid I, Uittenbogaart C, Jamieson B D. Live-cell assay for detection of apoptosis by dual-laser flow cytometry using Hoechst 33342 and 7-amino-actinomycin D. Nat Protoc 2007; 2(1):18790.
[27] Chand S, Mehta N, Bahia M S, Dixit A, Silakari O. Protein kinase C-theta inhibitors: a novel therapy for inflammatory disorders. Curr Pharm Des 2012; 18(30):472546.
[28] Martelli M P, Lin H, ZhangW, Samelson L E, Bierer B E. Signaling via LAT (linker for T cell activation) and Syk/ZAP70 is required for ERK activation and NFAT transcriptional activation following CD2 stimulation. Blood 2000; 96(6):2181890.
[29] Chi H, Flavell R A. Studies on MAP Kinase signaling in the immune system. Methods Mol Biol 2010; 661:471980.
[30] Chi G, Wei M, Xie X, Soromou L W, Liu F, Zhao S. Suppression of MAPK and NF-κB pathways by limonene contributes to attenuation of lipopolysaccharide-induced inflammatory responses in acute lung injury. Inflammation 2013; 36(2):50111.
[31] Salvador V H, Lima R B, dos Santos W D, Soares A R, Bohm P A F, Marchiosi R, et al. Cinnamic acid increases lignin production and inhibits soybean root growth. PLoS One 2013; vol. 8(7):e69105.
[32] Sticklen M B. Plant genetic engineering for biofuel production: towards affordable cellulosic ethanol. Nat Rev Genet 2008; 9(6):433243.
[33] Yi B, Hu L, Mei W, Zhou K, Wang H, Luo Y, et al. Antioxidant phenolic compounds of cassava (Manihot esculenta) from Hainan. Molecules 2011; 16(12):10157367.
[34] Meerungrueang W, Panichayupakaranant P. Antimicrobial activities of some Thai traditionalmedical longevity formulations fromplants and antibacterial compounds from Ficus foveolata. Pharm Biol 2014; 59(9):110449.
[35] Isakov N, Altman A. Protein kinase C(theta) in T cell activation. Annu Rev Immunol 2002; 20:761094.
[36] Nagy Z S, LeBaron M J, Ross J A, Mitra A, Rui H, Kirken R A. STAT5 regulation of BCL10 parallels constitutive NFkappaB activation in lymphoid tumor cells. Mol Cancer 2009; 8:67.
[37] Mitra A, Ross J A, Rodriguez G, Nagy Z S, Wilson H L, Kirken R A. Signal transducer and activator of transcription 5b (Stat5b) serine 193 is a novel cytokine-induced phospho-regulatory site that is constitutively activated in primary hematopoietic malignancies. J Biol Chem 2012; 287:165967608.
[38] Lutz-Nicoladoni C, Christina L-N, Thuille N, Nikolaus T, Wachowicz K, Katarzyna W, et al. PKCα and PKCβ cooperate functionally in CD3-induced de novo IL-2 mRNA transcription. Immunol Lett 2013; vol. 151 (no. 1, no. 2):318.
[39] Langlet C, Springael C, Johnson J, Thomas S, Flamand V, Leitges M, et al. PKC-alpha controls MYD88-dependent TLR/IL-1R signaling and cytokine production in mouse and human dendritic cells. Eur J Immunol 2010; 40(2):505915.
[40] Gruber T, Hermann-Kleiter N, Pfeifhofer-Obermair C, Lutz-Nicoladoni C, Thuille N, Letschka T, et al. PKC theta cooperates with PKC alpha in alloimmune responses of T cells in vivo. Mol Immunol 2009; 46(10):207109.
[41] Huang D, Zhou T, Lafleur K, Nevado C, Caflisch A. Kinase selectivity potential for inhibitors targeting the ATP binding site: a network analysis. Bioinformatics 2010; 26(2):198204.
[42] Liou J S, Chen J S, Faller D V. Characterization of p21Ras-mediated apoptosis induced by protein kinase C inhibition and application to human tumor cell lines”. J Cell Physiol 2004; vol. 198(2):27794.
[43] Couldwell W T, Hinton D R, He S, Chen T C, Sebat I, Weiss M H, et al. Protein kinase C inhibitors induce apoptosis in human malignant glioma cell lines. FEBS Lett 1994; 345(1):436.
[44] Querfeld C, Rizvi M A, Kuzel T M, Guitart J, Rademaker A, Sabharwal S S, et al. The selective protein kinase C beta inhibitor enzastaurin induces apoptosis in cutaneous T cell lymphoma cell lines through the AKT pathway. J Invest Dermatol 2006; vol. 126(7):1641o7.
[45] Yang X O, Nurieva R, Martinez G J, Kang H S, Chung Y, Pappu B P, et al. Molecular antagonism and plasticity of regulatory and inflammatory T cell programs. Immunity 2008; 29(1):44056.
[46] He X, Koenen HJPM, Smeets R L, Keijsers R, van Rijssen E, Koerber A, et al. Targeting PKC in human T cells using sotrastaurin (AEB071) preserves regulatory T cells and prevents IL-17 production. J Invest Dermatol 2014; 134(4):975683.
[47] deWeerd A, Kho M, Kraaijeveld R, Zuiderwijk J, WeimarW, Baan C. The protein kinase C inhibitor sotrastaurin allows regulatory T cell function. Clin Exp Immunol 2014; 175(2):2961304.

What is claimed is:

1. A method for preventing or treating Th1- or Th2-mediated immune disease, comprising a step of administering 4H3MC (4-hydroxy-3-methoxycinnamaldehyde) to a subject.

2. The method of claim 1, wherein the 4H3MC inhibits T-cell activity.

3. The method of claim 1, wherein the 4H3MC inhibits interleukin-2 (IL-2) production of T cells.

4. The method of claim 1, wherein the 4H3MC inhibits PKC (protein kinase C) activity of T cells.

5. The method of claim 1, wherein the Th1-mediated immune disease is transplant rejection, autoimmune disease or inflammatory disease.

6. The method of claim 1, wherein the Th1-mediated immune disease is selected from among colitis, inflammatory bowel disease, type 1 diabetes, type 2 diabetes, rheumatoid arthritis, reactive arthritis, osteoarthritis, psoriasis, scleroderma, osteoporosis, atherosclerosis, myocarditis, endocarditis, pericarditis, cystic fibrosis, Hashimoto thyroiditis, Graves' disease, Hansen's disease, syphilis, Lyme disease, borreliosis, neuroborreliosis, tuberculosis, sarcoidosis, lupus, discoid lupus, chilblain lupus, lupus nephritis, systemic Lupus erythematous, asthma, macular degeneration, uveitis, irritable bowel syndrome, Crohn's disease, Sjögren's syndrome, fibromyalgia, chronic fatigue syndrome, chronic fatigue/immune dysfunction syndrome, myalgic encephalomyelitis, amyotrophic lateral sclerosis, Parkinson's disease, multiple sclerosis, autism spectrum disorder, attention deficit disorder, and attention-deficit hyperactivity disorder.

7. The method of claim 1, wherein the Th2-mediated immune disease is allergic disease.

8. The method of claim 7, wherein the allergic disease is atopic skin disease.

9. A method for preventing or alleviating Th1- or Th2-mediated immune disease, comprising a step of administering to a subject a food composition comprising 4H3MC (4-hydroxy-3-methoxycinnamaldehyde).

10. The method of claim 9, wherein the Th2-mediated immune disease is allergic disease.

11. The method of claim 10, wherein the allergic disease is atopic skin disease.

12. A method for preventing or alleviating Th1- or Th2-mediated immune disease, comprising a step of applying to the skin a cosmetic composition comprising 4H3MC (4-hydroxy-3-methoxycinnamaldehyde).

13. The method of claim 12, wherein the Th2-mediated immune disease is allergic disease.

14. The method of claim 13, wherein the allergic disease is atopic skin disease.


SEQUENCE LISTING (4 sequences)

SEQ ID NO: 1 - DNA, 19 bp, Artificial
  a primer for PCR of human IL2
cacgtcttgcacttgtcac

SEQ ID NO: 2 - DNA, 21 bp, Artificial
  a primer for PCR of human IL2
ccttcttgggcatgtaaaact

SEQ ID NO: 3 - DNA, 24 bp, Artificial
  a primer for PCR of human GAPDH
cggagtcaacggatttggtcgtat

SEQ ID NO: 4 - DNA, 24 bp, Artificial
  a primer for PCR of human GAPDH
agccttctccatggtggtgaagac